(19) United States
(12) Patent Application Publication
(10) Pub. No.: US 2026/0263795 A1
(43) Pub. Date: Sep. 10, 2026

(54) WEARABLE EEG/EIT SYSTEM FOR MONITORING AND ENHANCING GLYMPHATIC CLEARANCE (GC) DURING SLEEP USING SKIN-PATH-CORRECTED SINGLE-FREQUENCY IMPEDANCE TO COMPUTE A GC INDEX WITH GC-WINDOW-GATED STIMULATION

(71) Applicant: Brain Electrophysiology Laboratory Company, LLC, Eugene, OR (US)

(72) Inventor: Don M. Tucker, Eugene, OR (US)

(21) Appl. No.: 19/654,066

(22) Filed: Apr. 21, 2026

Related U.S. Application Data
(62) Division of application No. 19/350,041, filed on Oct. 5, 2025.

(51) Int. Cl. A61N 1/36; A61B 5/00

(52) U.S. Cl. CPC A61N 1/36031; A61B 5/4088; A61B 5/7275; A61N 1/36025; A61N 1/36034

(57) ABSTRACT

A system for electrical stimulation and recovery of impressed currents during sleep to measure the electrical impedance of intracranial tissue through a single-frequency current stimulation, and decrease the brain impedance through other stimulation parameters, thereby increasing extracellular space and improving glymphatic flow (as indexed dynamically by the concurrent brain impedance measure). A skin-path-correction factor is estimated to allow the subtraction of the electrode-to-skin impedance and thereby estimate the brain impedance compartment separately. Based on computational modeling of electrical conductivity of head tissues, the electrodes are placed at forehead and nuchal sites to optimize current flow through high-conductive skull orifices. Current flow estimation is monitored at the critical orifice of the foramen magnum, and safety limits are monitored and enforced for individual electrodes and for key brain structures.

### RELATED APPLICATION

This application is a divisional of U.S. patent application Ser. No. 19/350,041, filed Oct. 5, 2025, which is incorporated by reference herein in its entirety.

### FIELD

The disclosure relates to wearable neuromonitoring and neuromodulation. In particular, it concerns integrated electroencephalography (EEG) and electrical impedance tomography/spectroscopy (EIT/EIS) to monitor cerebrospinal fluid-interstitial fluid exchange and to enhance glymphatic clearance during sleep, using single-frequency impedance with skin-path correction and window-gated transcranial electrical stimulation (tES).

### BACKGROUND

Extracellular space (ECS) fraction and fluid content modulate low-frequency tissue conductivity, and they also determine clearance of waste metabolites from the brain Glymphatic Clearance (GC). Wearable systems that sample a single-frequency intracranial impedance signal during sleep offer a practical proxy for ECS dynamics but are confounded by high and variable skin-electrode impedances. A need remains for an integrated EEG/EIT approach that (i) estimates an intracranial impedance component while correcting skin-path contributions; (ii) fuses the impedance signal with sleep stage to identify glymphatic-favorable windows; and (iii) optionally delivers tES within those windows under anatomical current-routing and safety constraints.

### STATEMENT REGARDING PRIOR ART

References such as WO 2018/039602 A1, U.S. Pat. No. 11,426,577 B2, and U.S. Pat. No. 12,005,251 B2 disclose head-mounted stimulation using burst patterns in which rest intervals exceed stimulation intervals with vascular/BOLD alternation endpoints. The present disclosure differs by (i) quantifying foramen-magnum routing via FM-COI; (ii) using continuous or symmetric-duty stimulation with quiescent or rest intervals not exceeding stimulus block duration; (iii) continuously or regularly acquiring a single-frequency intracranial impedance signal and combining it with Convolutional Neural Net (CNN) sleep staging to detect GC windows and optimize stimulation while preserving sleep architecture.

### SUMMARY

In one aspect, a wearable device places an active stimulation electrode at a suboccipital site and a return electrode on the forehead to bias current into the posterior fossa. An EEG front end electronics and a single-frequency impedance front end (e.g., ~7-8 Hz, nanoamp AC injection) operate continuously or at regular intervals during sleep. One or more processors obtain a head model to compute a Foramen-Magnum Current-Occupancy Index (FM-COI) and enforce field/skin safety caps; classify sleep with a convolutional neural network (CNN); compute a Global and/or Regional Glymphatic-Clearance Index (GCI/RGCI) from an intracranial-impedance/ECS estimate that is explicitly corrected for skin paths; detect glymphatic-clearance (GC) windows (increased ECS, decreased impedance); and schedule stimulation within GC windows while preserving sleep architecture.

Skin-path-corrected intracranial impedance (SPC-Z) may be estimated from electrode pairs that traverse brain tissue by subtracting a weighted combination of nearby “skin-only” control pairs (e.g., same-side frontal-mastoid and mastoid-mastoid) and/or by regression, adaptive filtering, or model-based inversion. Stage-conditioned and subject-specific parameters (weights, priors) can be learned from historical nights.

### DEFINITIONS

Single-frequency EIT channel: An impedance acquisition at a single frequency (such as ~7-8 Hz) using low-amplitude AC injection (such as nanoamperes), operated continuously or intermittently during sleep.
SPC-Z: Skin-path-corrected intracranial impedance: an estimate of intracranial impedance derived by suppressing electrode-gel-skin and scalp conduction contributions using control pairs and/or model-based terms.
GCI/RGCI: Global/Regional Glymphatic-Clearance Index: a scalar or vector metric computed from SPC-Z and regional sensitivities/priors (due to regional head tissue conductivity), summarizing clearance propensity across the night or within GC windows.
GC window: A time window predicted from sleep stage and SPC-Z signatures to favor CSF-ISF exchange and clearance.
FM-COI: Foramen-Magnum Current-Occupancy Index: a model-based ratio quantifying the fraction of injected current routed through a foramen-magnum region of interest.
Sleep-architecture safety: Constraints to preserve sleep structure (e.g., a REM, Rapid Eye Movement sleep, floor and limits on N3, deep Non-REM stage 3, fragmentation) in addition to electrical safety caps.

### BRIEF DESCRIPTION OF THE DRAWINGS

FIG. 1: Example position of tES/EEG/EIT electrodes in relation to head and skull anatomy. 10: Forehead electrode. 20: Nuchal (suboccipital) electrode.

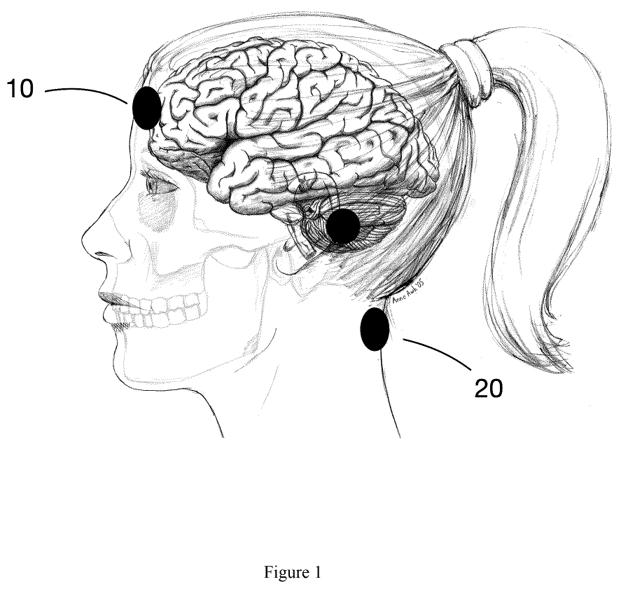

FIG. 2: Relationship of major system components. 30: Electrode positions as placed by a headband for EEG/tES/EIT. 40: Convolutional Neural Net machine learning operating on EEG to classify neurophysiological sleep stages. 50: Closed-loop controller for staging tES optimally in relation to sleep stages.

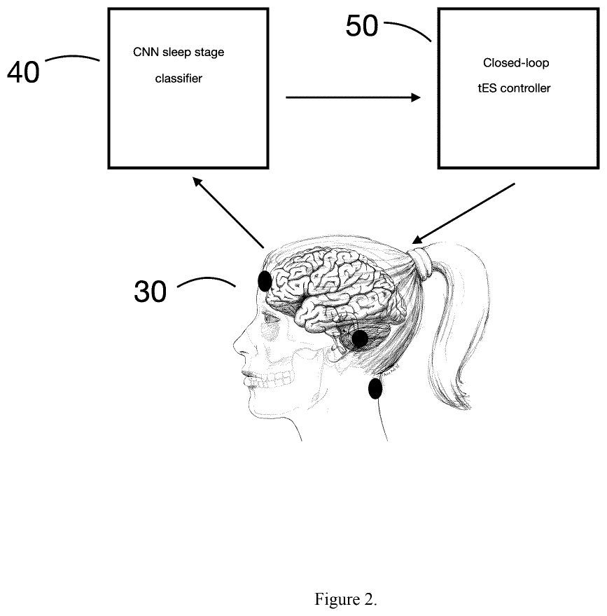

FIG. 3: Illustration of typical sleep stages in a chart described as a hypnogram, including wake (W), Rapid Eye Movement sleep (REM), Non-REM stage 1 (N1), Non-REM stage 2 (N2), and Non-REM stage 3 (N3). The early night is dominated by Non-REM and the later night by REM.

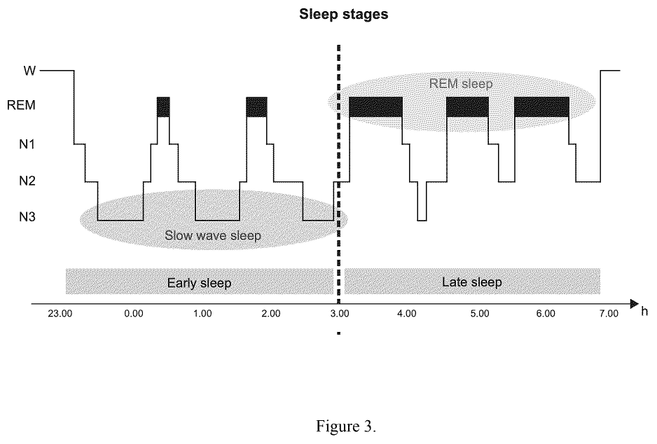

### DETAILED DESCRIPTION

### System Overview

A flexible wearable with a suboccipital (nuchal) electrode contact for tES current delivery and a forehead return. The controller hosts EEG, a single-frequency impedance channel, and a stimulation front end capable of continuous or symmetric-duty waveforms (e.g., 0.1-2 Hz slow-oscillation tES). The impedance channel runs continuously or at scheduled intervals to produce an impedance time series synchronized with EEG (electroencephalography).

### Skin-path Correction (SPC-Z)

The electrode-skin interface is a source of impedance that must be removed to estimate brain impedance (Z) correctly. To estimate Skin-Path Corrected brain impedance, SPC-Z, the processor forms brain-inclusive paths (e.g., forehead→contralateral mastoid; midline→mastoid) and suppresses skin contributions using proximal control pairs (e.g., forehead→forehead; mastoid→mastoid; same-side forehead→mastoid). One implementation subtracts half the sum of two nearest skin controls from a brain-inclusive path. More generally, a parametric form Z\hat{Z}_brain=Z_meas−α·Z_skin1−β·Z_skin2−γ·Z_skin3 is fit by ridge regression or adaptive filtering, optionally stage-conditioned and subject-specific. The processor rejects poor-quality epochs (motion, low SNR), optionally removes a slow time-of-night drift, and produces SPC-Z features (level, slope, variance, stage-conditioned contrasts).

### GCI/RGCI Computation

Regional sensitivity maps from a subject-specific or atlas-based head model transform SPC-Z to compartment-level estimates (cortex, deep gray, cerebellum, cisterna magna, upper spinal subarachnoid space) where extracellular space (ECS) is known to be indexed by brain electrical impedance. GCI may be the volume-weighted sum of RGCI or a principal-component projection. GC windows are detected from stage labels and SPC-Z features; stimulation is gated to the windows while preserving sleep-architecture safety and electrical caps (hindbrain E-field, cranial-nerve exposure, skin current-density).

### Use Cases

1) Nightly monitoring: compute GCI/RGCI trajectories to assess clearance propensity across the night, including stage-specific minima and overnight slopes. 2) Closed-loop enhancement: schedule slow-oscillation tES during GC windows to optimize RGCI while preserving REM and N3 continuity. 3) Longitudinal risk scoring: aggregate nightly GCI/RGCI features to predict chronic impairment of clearance and stratify risk for cognitive decline.

### Examples

Example A—Skin-path-corrected impedance from Sleep WISP (Wireless Interface Sensor Pod; neurosom. net) montage: Using forehead (Fp1, Fp2), mastoids (M1, M2), and a midline common (COM), compute SPC-Z for Fp2→M1 as \hat{Z}_brain=|Fp2−M1|−½(|Fp2−Fp1|+|M1−M2|). Other brain-inclusive paths (Fp1→M2, COM→M1) are similarly corrected.
Example B—WISP with continuous impedance: Five×4-min 0.75 Hz blocks with quiescent intervals≤the immediately preceding block; compute RGCI in real time; preserve REM minutes above a floor; titrate amplitude within anatomical and electrical caps.
Example C—Training and calibration: Intermittent multi-frequency EIS with EEG blanking validates single-frequency mappings via Kramers-Kronig consistency and joint inversion; cohort archives (e.g., hdEEG/EIT) provide priors for impedance-to-compartment mappings and GC-window definitions.

1. A method of risk stratification comprising: computing nightly GCI/RGCI trajectories from SPC-Z over multiple nights; extracting features including overnight slope, stage-specific minima, windowed integrals, and variance; and classifying chronic glymphatic-clearance impairment or predicting dementia risk.

2. The method of claim 1, wherein the classifier is trained on longitudinal cohorts labeled by fluid or imaging biomarkers and/or clinical outcomes.
